(19) United States
(12) Patent Application Publication
(10) Pub. No.: US 2010/0081929 A1
(43) Pub. Date: Apr. 1, 2010

(54) POSITION TRACKING METHOD, POSITION TRACKING DEVICE, AND ULTRASONOGRAPH

(76) Inventor: Takao SUZUKI, Kanagawa (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(21) Appl. No.: 12/066,708

(22) PCT Filed: Sep. 12, 2006
(86) PCT No.: PCT/JP2006/318021

§ 371 Date: Mar. 13, 2008

(30) Foreign Application Priority Data
Sep. 14, 2005 (JP) 2005-266785

(51) Int. Cl. A61B 8/04; G01S 15/06

(52) U.S. Cl. 600/437; 367/99

(57) ABSTRACT

The location tracking method of the present invention is a method for tracking the motion of a measuring point on an object by repeatedly irradiating the object with waves and analyzing received signals that are based on the waves reflected from the object, and includes the steps of: (A) irradiating the object with a wave and calculating an initial phase curve showing a phase shift of a received signal in a wave propagation direction with respect to a reference signal, the received signal being based on the wave reflected; (B) setting an initial location in the propagating direction and assigning the measuring point to the initial location; (C) defining a displacement line, which passes the initial location on the initial phase curve and of which the gradient is calculated based on the frequency of the reference signal; and (D) irradiating the object with the waves multiple times, thereby calculating phase curves, each representing a phase shift of the received signal with respect to the reference signal, and defining the intersection between the displacement line and each phase curve as a location to which the measuring point has been displaced.

### TECHNICAL FIELD

The present invention relates to a method and apparatus for tracking the location of an object of measurement by repeatedly irradiating the object with waves and detecting the waves reflected from the object, and also relates to an ultrasonic diagnostic apparatus.

### BACKGROUND ART

An ultrasonic diagnostic apparatus is used to make a noninvasive checkup on a subject by irradiating him or her with an ultrasonic wave and analyzing the information contained in its echo signal. For example, a conventional ultrasonic diagnostic apparatus that has been used extensively converts the intensity of an echo signal into its associated pixel luminance, thereby presenting the subject's structure as a tomographic image. In this manner, the internal structure of the subject can be known.

Meanwhile, some people are attempting recently to track the motion of a subject's tissue more precisely and evaluate the strain and the elasticity, viscosity or any other physical (attribute) property of the tissue mainly by analyzing the phase of the echo signal.

Patent Document No. 1 discloses a method for tracking a subject's tissue highly precisely by calculating the magnitude of instantaneous displacement of a local region of the subject based on the phase difference of an ultrasonic echo signal to be transmitted and received at regular intervals and by summing the magnitudes of displacements together. Hereinafter, a method for tracking a subject's tissue as disclosed in Patent Document No. 1 will be described with reference to FIG. 1. Suppose ultrasonic pulses are transmitted toward the same location of a subject at regular intervals ΔT and received signals, generated by converting the resultant echo signals into electrical signals, are identified by y1(t) and y2(t), respectively, where t represents the receiving time when the transmitting time is zero. The distance x from the probe (which will be simply referred to herein as a “distance”) and the receiving time t, which is the amount of time it takes for the received signal to arrive since its transmitting time, satisfy the following Equation (1), provided that C is the sonic velocity.

t1=x/(C/2) (1)

The received signals y1(t) and y2(t), which are functions of time, can be converted into y1(x) and y2(x) that are functions of distance by using Equation (1). Suppose there is a measuring point at a distance X and the measuring point has displaced ΔX parallel to the propagation direction of an ultrasonic wave during an interval ΔT. According to Patent Document No. 1, to calculate the magnitude of this displacement ΔX of the measuring point during the interval ΔT, orthogonal detection is carried out on y1(X) and y2(X) using a reference signal that has a frequency f. If the phase difference obtained by the orthogonal detection is Δθ, the following Equation (2) is satisfied.

ΔX=−C·Δθ/4πf (2)

The location X′ of the measuring point after the interval ΔT has passed is given by adding the magnitude of displacement ΔX, given by Equation (2), to the original measuring point X as in the following Equation (3).

X′=X+ΔX (3)

By performing this calculation repeatedly, the location of the measuring point in the subject can be tracked. For example, if the received signal that has been transmitted and received next is y3(x), the location X″ of the measuring point in 2ΔT can be calculated by substituting the phase difference Δθ′ between y2(X′) and y3(X′) into Equation (2) and then substituting the resultant ΔX′ into Equation (3).

Patent Document No. 2 further develops the method of Patent Document No. 1 into a method of calculating the elasticity of a subject's tissue (e.g., an arterial vascular wall, in particular). According to this method, first, an ultrasonic wave is transmitted from a probe 101 toward the vascular wall 302 of a subject 304 as shown in FIG. 2(a). And the echo signals, reflected from measuring points A and B on the vascular wall 302, are analyzed by the method of Patent Document No. 1, thereby tracking the motions of the measuring points A and B. FIG. 2(b) shows the tracking waveforms TA and TB of the measuring points A and B along with an electrocardiographic complex ECG.

As shown in FIG. 2(b), the tracking waveforms TA and TB have the same periodicity as the electrocardiographic complex ECG, which shows that the artery dilates and shrinks in sync with the cardiac cycle of the heart. More specifically, when the electrocardiographic complex ECG has outstanding peaks called “R waves”, the heart starts to shrink, thus pouring blood flow into the artery and raising the blood pressure. As a result, the vascular wall is dilated rapidly. That is why soon after the R wave has appeared on the electrocardiographic complex ECG, the artery dilates rapidly and the tracking waveforms TA and TB rise steeply, too. After that, however, as the heart dilates slowly, the artery shrinks gently and the tracking waveforms TA and TB gradually fall to their original levels. The artery repeats such a motion cyclically.

The difference between the tracking waveforms TA and TB is represented as a waveform W showing a variation in thickness between the measuring points A and B. Supposing the maximum variation of the thickness variation waveform is ΔW and the reference thickness between the measuring points A and B during initialization (i.e., the end of the diastole) is Ws, the magnitude of maximum strain ε between the measuring points A and B is calculated by the following Equation (4).

ε=ΔW/Ws (4)

As this strain is caused due to the difference between the blood pressures applied to the vascular wall, the elasticity Er between the measuring points A and B is given by the following Equation (5), provided that ΔP is the blood pressure difference at this time.

Er=ΔP/ε=ΔP·Ws/ΔW (5)

Therefore, by measuring the elasticity Er for multiple spots on a tomographic image, an image representing the distribution of elasticities can be obtained. If an atheroma 303 has been created in the vascular wall 302 as shown in FIG. 2(a), the atheroma 303 and its surrounding vascular wall tissue have different elasticities. That is why if an image representing the distribution of elasticities is obtained, important information can be acquired in inspecting the attribute of the atheroma (e.g., how easily the atheroma may rupture, among other things).

Likewise, the viscosity μ disclosed in Non-Patent Document No. 1 can also be calculated by this measuring method. The viscosity μ, which is an important index representing an attribute of the vascular wall tissue, is given by the following equation.

P=μdε/dt+Eε
Patent Document No. 1: Japanese Patent Application Laid-Open Publication No. 10-5226
Patent Document No. 2: Japanese Patent Application Laid-Open Publication No. 2000-229078
Non-Patent Document No. 1: Hiroshi Kanai, edited by the Acoustical Society of Japan, “Spectral Analysis of Sounds and Vibrations”, Corona Publishing Co., Ltd., ISBN4-339-01105-3

### DISCLOSURE OF INVENTION

### Problems to be Solved by the Invention

According to the method disclosed in Patent Document No. 1, however, if echoes that have been received from multiple sources of reflection are superposed one upon the other, then the received signals have disturbed phases, thus making it impossible to track the measuring point accurately. Among other things, since a vital tissue scatters a wave received, sometimes the tracking cannot be done accurately and a tissue attribute such as the elasticity cannot be measured precisely for that reason.

An object of the present invention is to provide a method and apparatus for tracking the location of a measuring point accurately even in such a situation where echoes from multiple sources of reflection are superposed one upon the other and where the received signals have disturbed phases, and also provide an ultrasonic diagnostic apparatus that adopts such a method and apparatus.

### Means for Solving the Problems

A location tracking method according to the present invention is a method for tracking the motion of a measuring point that has been set on an object of measurement by repeatedly irradiating the object with waves and by analyzing received signals that are based on the waves reflected from the object. The method includes the steps of: (A) irradiating the object with a wave and calculating an initial phase curve showing a phase shift of a received signal in a propagation direction of the wave with respect to a reference signal, the received signal being based on the wave reflected; (B) setting an initial location in the wave propagating direction and assigning the measuring point to the initial location; (C) defining a displacement line, which passes the initial location on the initial phase curve and of which the gradient is calculated based on the frequency of the reference signal; and (D) irradiating the object with the waves a number of times, thereby calculating phase curves, each representing a phase shift of the received signal with respect to the reference signal, and defining the intersection between the displacement line and each said phase curve as a location to which the measuring point has been displaced.

In one preferred embodiment, the received signal is discrete quantized digital data, and the step (D) includes calculating the newest phase curve by figuring out a phase difference between the newest received signal and the signal that has been received the previous time and by adding the phase difference thus obtained to the previous phase curve.

In another preferred embodiment, the received signal is discrete quantized digital data, and the step (D) includes calculating the newest phase curve by figuring out a phase difference between the newest received signal and the signal that has been received the previous time at one of multiple sample points that have been set in the wave propagating direction, adding the phase difference thus obtained to a phase at the one sample point on the previous phase curve, and sequentially adding the phase difference of the newest received signal in a depth direction to the sum.

In still another preferred embodiment, the received signal is discrete quantized digital data, and the step (C) includes determining a phase at the initial location by subjecting sampled data values of multiple phases to interpolation.

In yet another preferred embodiment, the received signal is discrete quantized digital data, and the step (D) includes finding an intersection either between multiple approximation lines, obtained based on sampled data of multiple phases, or between an approximation curve and the displacement line.

Another location tracking method according to the present invention is a method for tracking the motion of a measuring point that has been set on an object of measurement by repeatedly irradiating the object with waves and by analyzing received signals that are based on the waves reflected from the object. The method includes the steps of: (A) irradiating the object with a wave to obtain a first received signal that is based on the wave reflected; (B) calculating a first phase curve showing a phase shift of the first received signal in a propagation direction of the wave with respect to a reference signal; (C) setting a first location in the wave propagating direction and assigning the measuring point to the first location; (D) defining a displacement line, which passes the first location on the first phase curve and of which the gradient is calculated based on the frequency of the reference signal; (E) irradiating the object with a wave, thereby calculating a second phase curve, representing a phase shift of the second received signal with respect to the reference signal; and (F) defining the intersection between the displacement line and the second phase curve as a second location to which the measuring point has been displaced. The location of the measuring point is tracked by performing the steps (B) through (E) all over again with the second received signal of the step (E) and the second location of the step (F) substituted for the first received signal and the first location, respectively.

In one preferred embodiment, the step (E) includes calculating the second phase curve by figuring out a phase difference between the first and second received signals and by adding the phase difference thus obtained to the first phase curve.

In another preferred embodiment, the step (E) includes calculating the second phase curve by figuring out a phase difference between the first and second received signals at one of multiple sample points that have been set in the wave propagating direction, adding the phase difference thus obtained to a phase at the one sample point on the first phase curve, and sequentially adding the phase difference of the second received signal in a depth direction to the sum.

In this particular preferred embodiment, the step (E) includes setting one of the sample points closest to the measuring point.

In a specific preferred embodiment, the step (E) includes calculating a portion of the second phase curve according to the sign of the phase difference between the first and second received signals and defining the intersection between the displacement line and that portion of the second phase curve as the location to which the measuring point has been displaced.

In still another preferred embodiment, the step (B) includes calculating the first phase curve based on only the first received signal.

In yet another preferred embodiment, the step (B) includes calculating a portion of the first phase curve by finding phases at multiple sample points that have been set in the wave propagating direction.

Still another location tracking method according to the present invention is a method for tracking the motion of a measuring point that has been set on an object of measurement by repeatedly irradiating the object with waves and by analyzing received signals that are based on the waves reflected from the object. The method includes the steps of: (A) irradiating the object with a wave to obtain a first received signal that is based on the wave reflected; (B) calculating a first phase curve showing a phase shift of the first received signal in a propagation direction of the wave with respect to a reference signal; (C) setting a first location in the wave propagating direction and assigning the measuring point to the first location; (D) defining a displacement line, which passes one of multiple sample points that have been set in the wave propagating direction and of which the gradient is calculated based on the frequency of the reference signal, the one sample point being located closest to the first location; (E) irradiating the object with a wave, thereby calculating a second phase curve, representing a phase shift of the second received signal with respect to the reference signal; and (F) finding the intersection between the displacement line and the second phase curve, adding the distance from the intersection to the sample point that is closest to the first location to the location of the measuring point to set a second location, and defining the second location as a location to which the measuring point has been displaced. The location of the measuring point is tracked by performing the steps (B) through (F) all over again with the second received signal of the step (E) and the second location of the step (F) substituted for the first received signal and the first location, respectively.

In one preferred embodiment, the step (E) includes the steps of: (E1) calculating the phase difference between the first and second received signals at the sample point that is located closest to the first location; (E2) adding the phase difference, calculated in the step (E1), to the phase at the closest sample point on the first phase curve, thereby calculating the phase of the second received signal at that closest sample point; (E3) calculating phase differences for multiple sample points that are adjacent to the closest sample point; and (E4) defining either an approximation line or an approximation curve as the second phase curve based on the phase that has been calculated in the step (E2) and the phase difference that has been calculated in the step (E3).

In yet another preferred embodiment, if the unit of the distance axis of a distance-phase plane is a length unit, the gradient of the displacement line is calculated −4πf/C, where f is the frequency of the reference signal and C is the propagation velocity of the waves.

In an alternative preferred embodiment, if the unit of the distance axis of a distance-phase plane is a receiving time unit, the gradient of the displacement line is calculated −2πf, where f is the frequency of the reference signal.

A location tracking apparatus according to the present invention includes: a transmitting section for irradiating the object with a wave; a receiving section for receiving a wave that has been reflected from the object to generate a received signal; and a tracking section for tracking the location of a measuring point that has been set on the object by any of the location tracking methods described above.

An ultrasonic diagnostic apparatus according to the present invention includes: a transmitting section for transmitting an ultrasonic wave toward an object of measurement using a probe; a receiving section for receiving a wave that has been reflected from the object through the probe to generate a received signal; and a tracking section for tracking the location of a measuring point that has been set on the object by any of the location tracking methods described above in cooperation with the transmitting and receiving sections. The apparatus evaluates at least one of the shape property and the attribute property of the object based on the location of the measuring point that has been calculated by the tracking section.

In one preferred embodiment, the attribute property is one of the magnitude of strain, elasticity and viscosity of the object.

In another preferred embodiment, the apparatus further includes an elasticity calculating section that receives a signal representing a variation in stress applied to the object and that calculates the elasticity of the object based on the location of the measuring point that has been detected by the tracking section.

In still another preferred embodiment, the object of measurement is a vascular wall and the signal representing the variation in stress is a blood pressure waveform.

In yet another preferred embodiment, the object of measurement is a vascular wall and the attribute property is a diagnostic index of arterial sclerosis including at least one of an intima-media thickness (IMT) and a pulse wave velocity (PWV).

In yet another preferred embodiment, the object of measurement is a heart and the shape property is a contraction/dilation property.

### EFFECTS OF THE INVENTION

According to the location tracking method of the present invention, even if a number of reflected waves that have come from multiple sources of reflection are received as superposed waves and if the phases of the received signals are disturbed, the motion of the measuring point that has been set in the object can also be tracked accurately. In addition, the ultrasonic diagnostic apparatus of the present invention can also be used to accurately measure an attribute of the subject's tissue such as the strain, elasticity or viscosity thereof and precisely evaluate the motion function of the subject's tissue in terms of the shrinkage and dilation thereof, for example.

### BRIEF DESCRIPTION OF DRAWINGS

FIG. 1 shows a method for tracking a tissue based on the phase difference of an ultrasonic echo signal.

FIG. 2(a) is a schematic representation showing how to calculate the elasticity of a vascular wall and FIG. 2(b) shows how to calculate the magnitude of strain using a tracking waveform obtained from a vascular wall.

FIG. 3 shows a conventional method for tracking the location of a measuring point based on a phase difference.

FIG. 4 shows a configuration for an orthogonal detector.

FIG. 5 shows a first preferred embodiment of a location tracking method according to the present invention.

FIG. 6 shows how phase curves change with time.

FIG. 7 is a flowchart showing the procedure of the location tracking method of the first preferred embodiment.

FIG. 8 shows how to correct a phase that is discontinuous in the distance direction.

FIG. 9 shows an offset between phase curves.

FIG. 10 shows an exemplary method of calculating a phase curve.

FIG. 11 shows another exemplary method of calculating a phase curve.

FIG. 12 shows an exemplary method of locating an intersection between a phase curve and a displacement line.

FIG. 13 is a flowchart showing the procedure of a location tracking method according to the present invention that adopts digital signal processing.

FIG. 14 is a flowchart showing the procedure of a location tracking method according to a second preferred embodiment of the present invention.

FIG. 15 illustrates how the procedure shown in FIG. 14 is carried out.

FIG. 16 is a flowchart showing the procedure of a simplified version of the location tracking method of the second preferred embodiment.

FIG. 17 illustrates how the procedure shown in FIG. 16 is carried out.

FIG. 18 is a flowchart showing the procedure of another simplified version of the location tracking method of the second preferred embodiment.

FIG. 19 illustrates how the procedure shown in FIG. 18 is carried out.

FIG. 20 is a block diagram showing the configuration of an ultrasonic diagnostic apparatus according to the present invention.

FIG. 21 shows an example of a picture presented on the monitor of the ultrasonic diagnostic apparatus shown in FIG. 20.

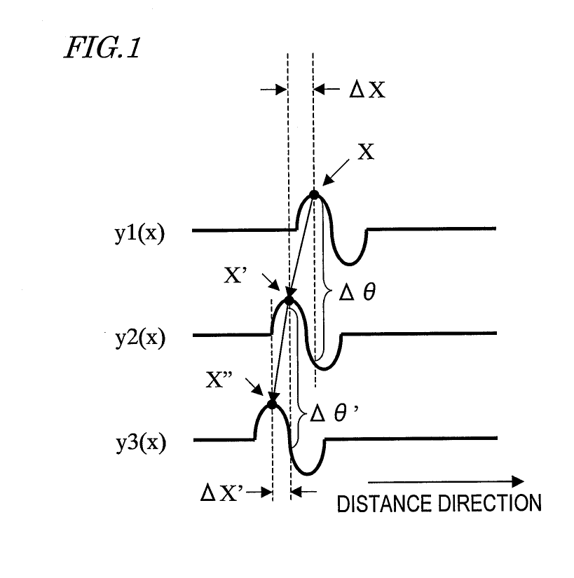

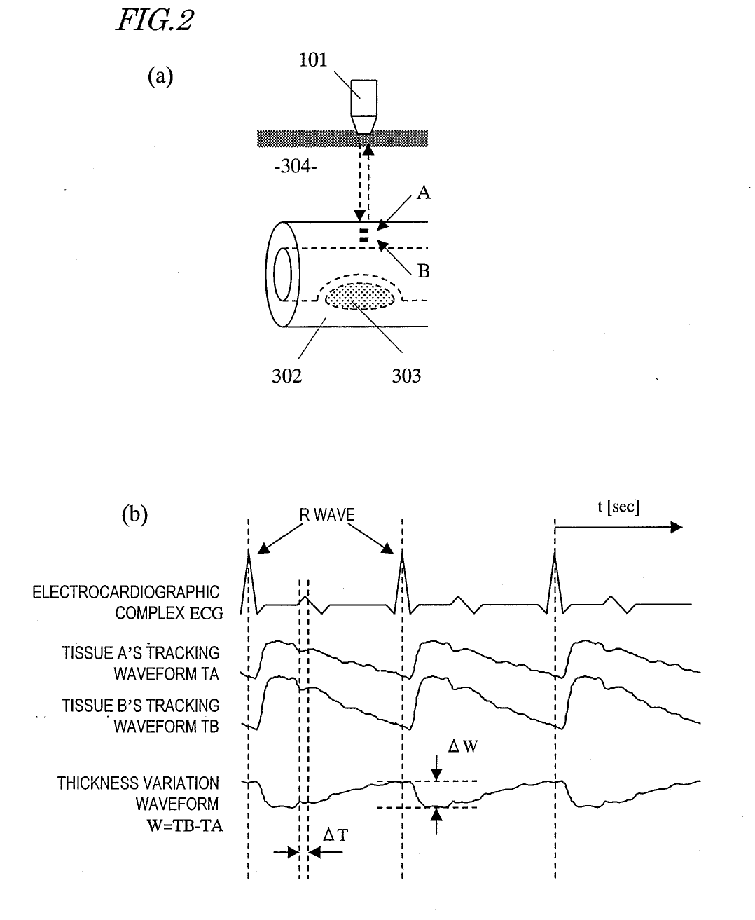

### DESCRIPTION OF REFERENCE NUMERALS

100 control section
101 probe
102 transmitting section
103 receiving section
104 tomographic image processing section
105 tissue tracking section
106 image synthesizing section
107 monitor
108 elasticity calculating section
111 blood pressure manometer
112 blood pressure manometer controlling and blood pressure value retrieving section
120 memory
121 memory
200 tomographic image
201 elasticity image
202 tomographic image reflection intensity scale
203 elasticity image scale
204 biomedical signal waveform

### BEST MODE FOR CARRYING OUT THE INVENTION

### Embodiment 1

First, the Problems of the Conventional Method disclosed in Patent Document No. 1 will be pointed out with reference to FIG. 3, which shows the results obtained by transmitting a wave (i.e., an ultrasonic wave in Patent Document No. 1) and by analyzing waves reflected by two point reflection sources that were arranged in the direction in which the transmitted wave propagated. The upper portion of FIG. 3 shows a received signal y1(x) obtained by transforming a received signal y1(t) into a function of distance by Equation (1). The received signal y1(t) was generated by converting a reflected one of a wave that had been transmitted at a time T=0 into an electrical signal. The middle portion of FIG. 3 also shows a received signal y2(t), obtained from the same point reflection sources by transmitting the wave all over again ΔT later, as a function of distance y2(x). The received signals y1(x) and y2(x) include wave trains w1 and w2, which are waves reflected from the two reflection sources, respectively. Since the interval between the two point reflection sources is shorter than the combined lengths of these wave trains, the wave trains w1 and w2 overlap with each other. Also, the two point reflection sources are supposed to be approaching the source of transmission and reception (i.e., a probe in Patent Document No. 1) at the same velocity. For that reason, if the transmitting time is supposed to be a reference time, the wave trains w1 and w2 in the received signal y2(x) are received earlier than the counterparts in the received signal y1(x).

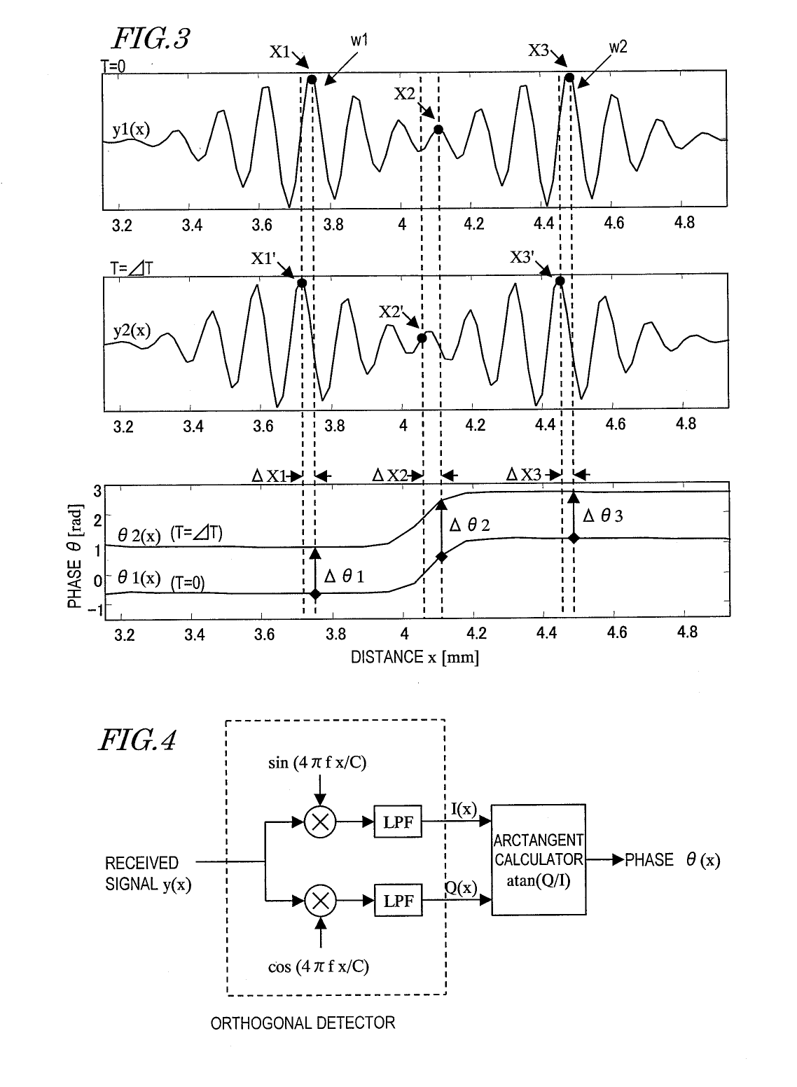

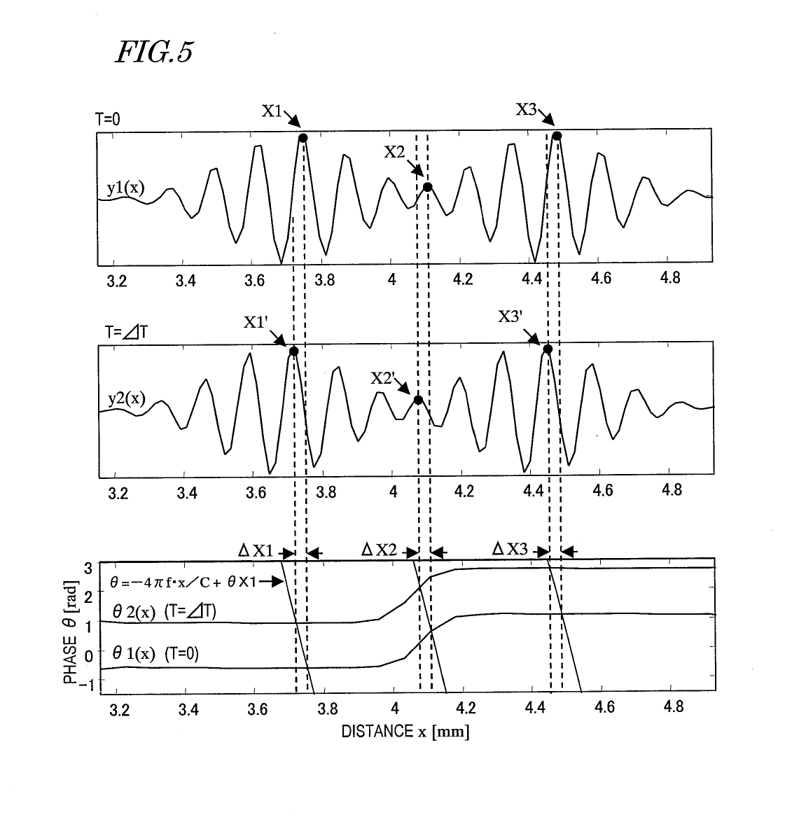

The lower portion of FIG. 3 shows phases θ1(x) and θ2(x) obtained by subjecting these two received signals y1(x) and y2(x) to orthogonal detection by using the detector shown in FIG. 4 and by calculating the arctangent of the complex received signals I(x) and Q(x), which are the outputs of the detector. As the reference frequencies of the orthogonal detection, the carrier frequencies of the received signals are used. Also, the phases obtained by the orthogonal detection are corrected by adding ±2 π (where n is a positive integer) thereto such that the phases θn(x) are continuous with each other in the distance direction. These θn(x) will be referred to herein as “phase curves”.

According to the method disclosed in Patent Document No. 1, the phase difference Lie between these two phase curves at the same distance is subjected to the calculation of Equation (2) to obtain the magnitude of displacement ΔX, which is then added to the previous location X as in Equation (3), thereby figuring out a location to be tracked. For example, a distance X1′ that a measuring point will travel in ΔT if a distance X1 is set for a received signal of the wave that has been transmitted at the time T=0 is calculated by substituting a phase difference Δθ1 at the distance X1 into Equation (2) to obtain the magnitude of displacement ΔX1 and by adding this magnitude of displacement ΔX1 to X1. A distance X3′ that a measuring point will travel in ΔT if a distance X3 is set for the received signal can also be figured out by performing similar calculations.

The magnitudes of displacement ΔX1 and ΔX3 of the measuring points at the distances X1 and X3 in the wave trains w1 and w3 shown in FIG. 3, where a single wave train prevails, can be calculated accurately by this method. That is to say, the locations of these measuring points can be tracked sequentially because the phases match with each other within the wave trains w1 and w2. However, where these two wave trains overlap with each other, a measuring point at a distance X2 that has been set for the received signal of a wave transmitted at the time T=0 in FIG. 3, for example, cannot be tracked accurately. This is because the degree of overlap between these two wave trains changes with the distance and therefore the phases are not flat and also because the displacement of an object in the wave propagating direction is not represented only by the increase and decrease of phases if the received signal is transformed into a distance-phase plane. For example, as shown in the lower portion of FIG. 3, the phase curve θ2(x) shifts not only upward but also leftward with respect to the phase curve θ1(x). That is why the phase difference Δθ2 is found to be greater than the phase differences Δθ1 and Δθ3, and the magnitude of displacement ΔX2 of the distance X2 is calculated a relatively large value. In FIG. 3, as the object is coming toward the source of transmission and reception, the phase curve θ2(x) shifts upper-leftward with respect to the phase curve θ1(x). If the object is going away from the source of transmission and reception, however, the phase curve θ2(x) will shift lower-rightward. In addition, since the phase of the wave train w2 is ahead of that of w1, the curve rises rightward in the lower portion of FIG. 3. On the other hand, if the phase of the wave train w2 is behind that of w1, the curve may fall rightward in the lower portion of FIG. 3. In that case, the magnitude of displacement ΔX2 is calculated a relatively small value. As described above, the measuring point at the distance X2 in the received signal of a wave that has been transmitted at the time T=0 cannot be tracked properly. That is to say, a tracking error is generated.

According to the present invention, such a tracking error can be reduced. Hereinafter, the location tracking method of the present invention will be described with reference to FIG. 5. The location tracking method of the present invention utilizes the fact that if the received signal is transformed into a distance-phase plane, the wave shift of the object in the transmitting and receiving direction is represented not just by the increase and decrease of the phase but also by those of the distance and that these variations are produced in conjunction with each other. More specifically, the magnitude of wave shift of the object in the transmitting and receiving direction during ΔT, i.e., the magnitude of shift between the wave trains of two received signals that have been transmitted and received at an interval of ΔT, and the phase shift during ΔT, obtained by subjecting the received signals to an orthogonal detection, satisfy the relation represented by Equation (2), which can be modified into the following Equation (6).

Δθ/ΔX=−4πf/C (6)

The upper, middle and lower portions of FIG. 5 show the received signal y1(x), the received signal y2(x) and the phase curves θ1(x), θ2(x), respectively, which are produced under the same conditions as in FIG. 3. The magnitude of shift between the received signals should satisfy the relation represented by Equation (6). Therefore, if the initial location of a measuring point is set on the received signal of a wave that has been transmitted at the time T=0, then the displacement of the measuring point at the distance X1 is represented by the line, which passes (X1, θ1(X1)) on the distance-phase plane shown in the lower portion of FIG. 5 and of which the gradient is given by Equation (6) (i.e., the line represented by the following Equation (7)). Thus, the displacement of the measuring point X1 at the distance X1 is represented by the line given by the following Equation (7).

θ=−4πf(x−X1)/C+θ1(X1) (7)

In the Equation (7), f is the frequency of a reference signal used in orthogonal detection. This line will be referred to herein as a “displacement line”.

Thus, the distance X1′ that the measuring point travels in the wave propagating direction in ΔT is the distance at the intersection between the displacement line given by Equation (7) and the phase curve θ2(x). The distance X″ of the next destination is the distance at the intersection between the displacement line given by Equation (7) and a phase curve θ3(x) (not shown). The same statement applies to distances X2 and X3, too. As can be seen FIG. 5, the peak portions of the received signal waveform can be tracked accurately irrespective of the degree of overlap between the wave trains.

According to the conventional method, the magnitude of displacement is calculated by Equation (2) using the phase difference at the same distance. In that case, however, if the waves overlapped with each other to disturb phases, the magnitude of displacement would contain some errors. On the other hand, according to the location tracking method of the present invention, an initial location is set, and a displacement line representing the displacement of the initial location as given by Equation (7) is plotted on a distance-phase plane defining a phase curve. For that reason, even if the waves overlap with each other to disturb the phases, the location of the measuring point displaced can be calculated as the intersection between the phase curve and the displacement line.

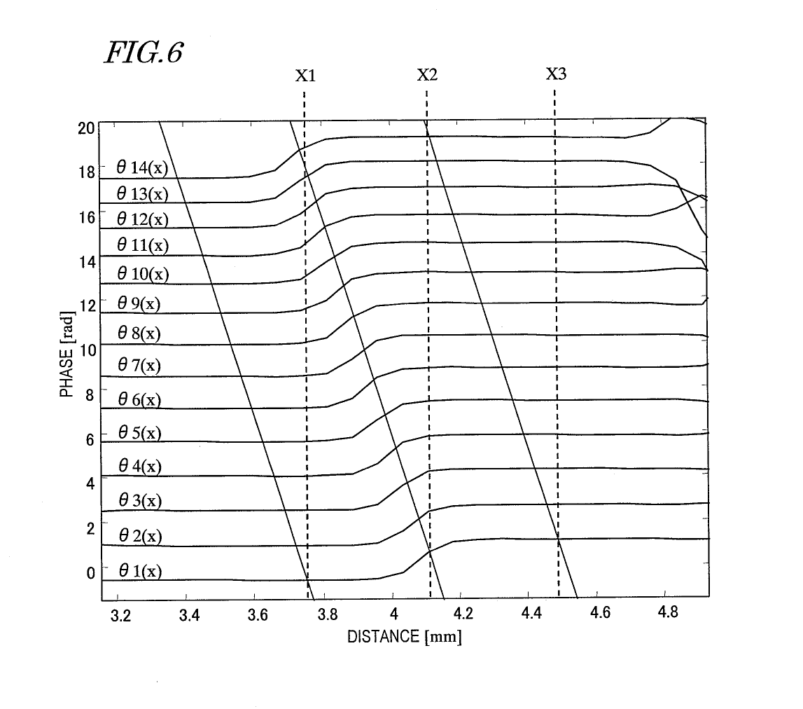

FIG. 6 shows the shifts of phase curves θn(x) in a situation where the source of reflection is coming toward the source of transmission and reception at a constant velocity under the same condition as in FIG. 3 and where the waves are transmitted and received at an interval of ΔT. FIG. 6 also shows the displacement lines of measuring points in a situation where initial locations are set at distances X1, X2 and X3. The initial location of a measuring point is set by the distance and phase that are defined on the phase curve θ1(x) at the transmitting time T=0. As can be seen from the line representing the displacement of the measuring point set at the distance X2, the oblique phases can be tracked accurately, and therefore, overlapping portions between the waves of received signals can be tracked. Thus, according to the location tracking method of the present invention, even if mutually overlapping echoes are received from multiple sources of reflection and if the phases of the received signals are disturbed, the displacement of the measuring point can also be tracked accurately.

Also, according to the location tracking method of the present invention, the magnitudes of displacements are not added together one after another unlike the method disclosed in Patent Document No. 1, and therefore, the errors are never accumulated at the location to be tracked. The reference frequency f of the orthogonal detection is usually set in the vicinity of the center frequency of a transmitted pulse or the carrier frequency of a received signal. However, according to the present invention, the reference frequency f can be set freely and does not have to be the vicinity of the center frequency of a transmitted pulse or the carrier frequency of a received signal. Furthermore, according to the present invention, the phase just needs to be calculated for a reference signal with a single predetermined frequency f and the phase detection means does not have to be orthogonal detection. For example, the phase may also be figured out by a Fourier transform with the regions divided. In Equation (7), the unit in the distance direction is supposed to be a length unit. Alternatively, the unit may also be a time unit that is defined by Equation (1) with respect to the transmitting time. In that case, if the initial location is t1, the displacement line given by Equation (7) may be represented by the following Equation (8).

θ=−2πf(t−t1)+θ1(t1) (8)

Hereinafter, a specific procedure of the location tracking method of the present invention will be described with reference to FIGS. 5, 7, 8 and 9. First, in Step S301, an object is irradiated with a wave and the wave reflected from the object is converted into an electrical signal, thereby obtaining a received signal y1(x). Next, in Step S302, the received signal y1(x) is subjected to an orthogonal detection using a reference signal with a single frequency f to find the phase of the received signal y1(x) with respect to the reference signal. Subsequently, in Step S303, the arctangent of the resultant complex received signal is calculated, thereby figuring out a phase curve θ1(x) representing its phase in the wave propagating direction with respect to the reference signal. The phase curve obtained in this process step may be discontinuous between −π and π as shown in FIG. 8. That is why as indicated by the dashed line in FIG. 8, ±2π is added, thereby correcting the phase curve into a continuous one. Also, to track the location of the object, the object is repeatedly irradiated with a plurality of waves to figure out multiple phase curves as will be described later. For that reason, ±2nπ (where n is a positive integer) is added to the entire phase curve such that a phase curve and its previous phase curve have a phase difference approximately within ±π as shown in FIG. 9. If the previous phase curve is θ1(x) as shown in FIG. 9, the phase curve θ2′(x) to be obtained now should have a phase difference of at least π with respect to θ1(x). That is why −2π is added to (i.e., 2π is subtracted from) the entire θ2′(x) to obtain a proper phase curve θ2(x).

Next, in Step S304, it is determined whether the location of the measuring point should be initialized or not. More particularly, if a signal instructing that the tracking location of the measuring point be initialized has been detected, then the measuring point is assigned to an initial location in the received signal in Step S307. Even if the received signal is the first one that has ever been received, the processing step S307 is also carried out in response to that signal instructing that the tracking location be initialized. Either a single measuring point or a plurality of measuring points may be set. For example, in FIG. 5, initial locations X1, X2 and X3 are set at distances of approximately 3.75 mm, 4.1 mm and 4.5 mm, respectively, and measuring points are assigned to all of these three locations.

Thereafter, in Step S308, a displacement line (given by Equation (7)) that passes the initial location on the phase curve θ1(x) and that is defined by the frequency f of the reference signal and the propagation velocity C of the wave is determined. In FIG. 5, for example, three lines that pass the initial locations X1, X2 and X3 on the phase curve θ1(x) and that have a gradient −4πf/C are determined.

Next, the processing steps S301, S302 and S303 are carried out all over again. Specifically, the object is irradiated with another wave, a received signal is generated based on the wave reflected, and a phase curve θ2(x) representing the phase of the received signal in the wave propagating direction with respect to the reference signal is obtained. Unless the signal instructing that the tracking location of the measuring point be initialized has been received, the process advances from Step S304 to Step S305, in which the intersection between the phase curve θ2(x) just obtained and the displacement line is located. Then, in Step S306, the intersection found is defined as the destination of the measuring point. That is to say, the new location of the measuring point is determined.

After that, the same processing steps S301, S302, S303, S304, S305 and S306 are carried out all over again to irradiate the object with another wave, obtain another phase curve, find an intersection between a displacement line and the phase curve, and locate the measuring point repeatedly.

Also, the signal instructing that the tracking location of the measuring point be initialized is output at an appropriate interval. Thus, every time the signal instructing that the tracking location of the measuring point be initialized is detected, the location of the measuring point is initialized and a displacement line that passes the initial location is obtained.

In measuring the elasticity of a vascular wall as disclosed in Patent Document No. 2, the signal instructing that the tracking location of a measuring point be initialized may be a pulse sync signal represented by R waves of an electrocardiograph (ECG), for example. Also, according to Patent Document No. 2, multiple initial locations of measuring points are set at regular intervals in the distance direction, which is on the acoustic line of the ultrasonic wave. By repeatedly performing such a procedure, the subject's tissue can be tracked accurately.

Hereinafter, a specific phase curve calculating method will be described. If the location is tracked by the location tracking method of the present invention, an actual apparatus may sample the received signal and process it as a digital signal either before or after the orthogonal detection. For that reason, the resultant phase curve will not be a continuous curve but actually a group of phase data representing the phase of the received signal with respect to the reference signal at multiple sample points that have been set at predetermined intervals in the wave propagating direction. In general, those sample points are fixed and never move while the location of the object is being tracked.

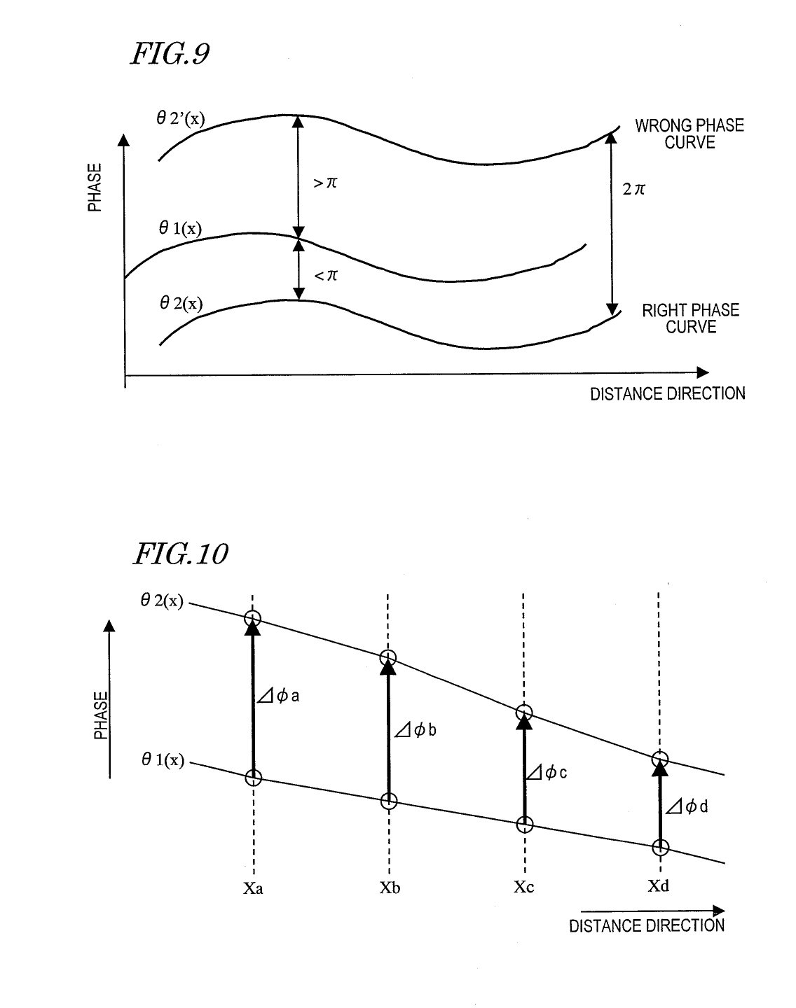

First, as shown in FIG. 10, if a signal instructing that the tracking location of the measuring point be initialized has been detected or if the phase curve θ1(x) should be calculated for the signal that has been received for the very first time, the received signal is subjected to an orthogonal detection and the arctangent of the resultant complex received signal is calculated, thereby finding the phases of the received signal with respect to sample points Xa, Xb, Xc and Xd. In FIG. 10, the open circles ◯ on the phase curve θ1(x) indicate the sample points. As described above, the phase curve θ1(x) actually has data only at those sample points, i.e., is actually a group of phase data.

To obtain a phase curve θ2(x) based on a signal that has been received for the second time, phase differences Δφa, Δφb, Δφc and Δφd between the phases θ1(Xa), θ1(Xb), θ1(Xc) and θ1(Xd) of the first group of phase data, calculated based on the first received signal and representing a phase curve, and the phases of the second received signal are figured out. The phase differences can be figured out by calculating the arctangent of the complex conjugate product of the first and second complex received signals that have already been subjected to the orthogonal detection. Next, as pointed by the arrows in FIG. 10, the phase differences Δφa, Δφb, Δφc and Δφd thus obtained are added to the phases θ1(Xa), θ1(Xb), θ1(Xc) and θ1(Xd) of the first group of phase data that have been calculated based on the first received signal and that represent a phase curve. As a result, a second group of phase data, representing a phase curve θ2(x) consisting of θ2(Xa), θ2(Xb), θ2 (Xc) and θ2(Xd), can be obtained. In this processing step, if the displacement of the object is partially quick, then the phase may become discontinuous between −π and π. In that case, ±2π is added to correct the phase into a continuous one as already described with reference to FIGS. 8 and 9. Likewise, to obtain a third group of phase data, the phase differences between the second and third received signals are added to the second group of phase data. The same statement applies to the other groups of phase data, too.

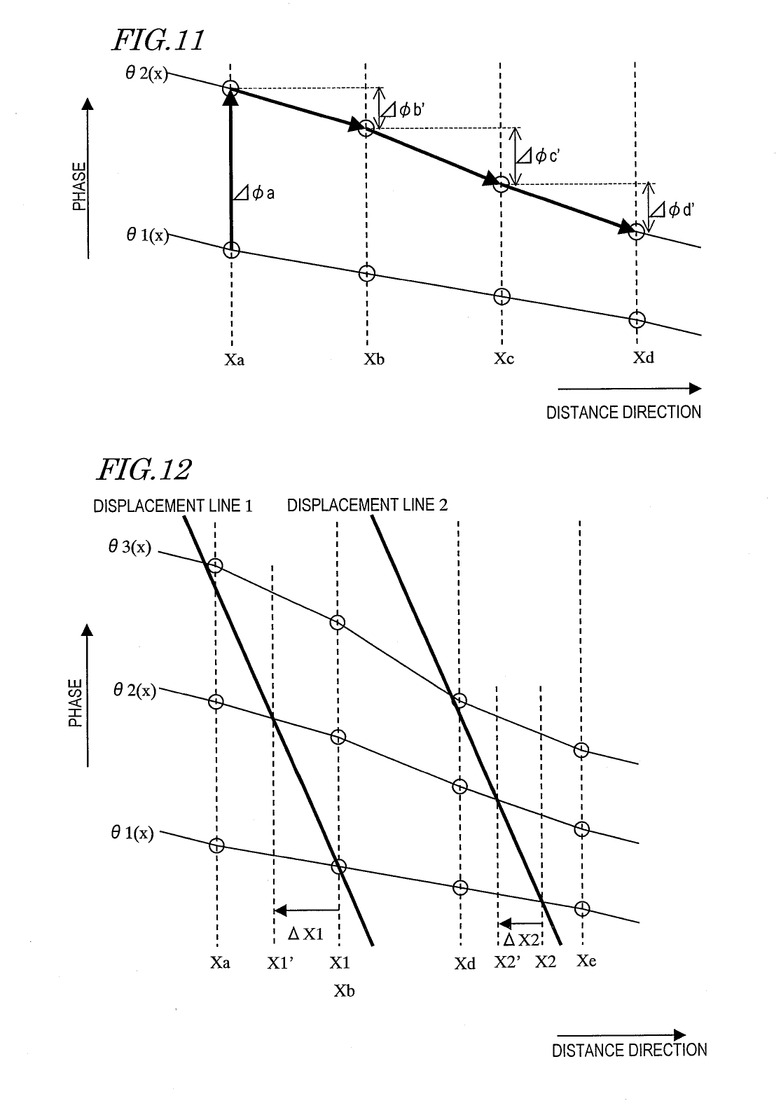

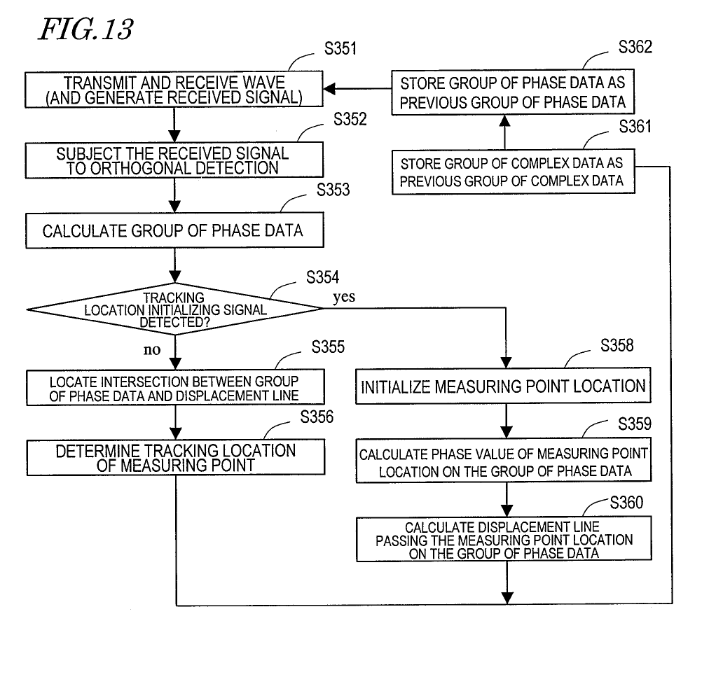

Alternatively, the phase curve θ2(x) may also be figured out by another method. To figure out a phase curve θ2(x) based on the second received signal, a phase difference between the phase at any of the sample points of the first group of phase data obtained based on the first received signal and the phase at that sample point on the second received signal is calculated as shown in FIG. 11. For example, a phase difference Δφa is calculated at a sample point Xa. Next, the phase difference Δφa is added to the phase θ1(Xa) of the first group of phase data at the sample point Xa, thereby obtaining the phase θ2(Xa) at the sample point Xa of the phase curve θ2(x) based on the second received signal.

Subsequently, phase differences Δφb′, Δφc′ and Δφd′ between two adjacent samples points on the phase curve θ2(x) are calculated and are sequentially added to the phase θ2 (Xa) at the sample point Xa on the phase curve θ2(x), thereby obtaining a second group of phase data showing the phase curve θ2(x) that consists of θ2(Xa), θ2(Xb), θ2(Xc) and θ2(Xd). Likewise, to obtain a third group of phase data, a phase difference between the second and third received signals is calculated at any of the sample points, third phase data at that point is obtained, and then the phase difference between adjacent sample points of the third received signal is added, thereby calculating the third group of phase data. The same statement applies to the other groups of phase data.

Next, an exemplary method of locating the intersection between a phase curve and a displacement line will be described with reference to FIG. 12. As described above, the phase curve is actually a group of phase data consisting of only the phases at sample points. If the signal instructing that the tracking location of a measuring point be initialized is detected when the phase curve θ1 is calculated, measuring points are set at initialized locations X1 and X2. In this case, if the initialized location (e.g., X1) agrees with one of the sample points, a displacement line #1 that passes (X1, θ1(X1)) on a distance-phase plane is calculated.

On the other hand, if the initialized location (e.g., X2) does not agree with any of the sample points, then a line is drawn tentatively on the supposition that the phase changes linearly between the previous and following sample points Xd and Xe (shown on the left- and right-hand sides of X2 in FIG. 12), and the phase at the X2 location on that line is obtained, thereby calculating a phase line #2 that passes the location on a distance-phase plane.

After a group of phase data representing the phase curve θ2(x) has been obtained based on the next received signal, the intersection between the displacement line and the phase curve θ2(x) is located. Since the sample points are not continuous on the phase curve θ2(x) in this case, the intersection between the phase curve and the displacement line does not agree with any of the sample points in most cases. That is why a line is tentatively drawn on the supposition that the phase changes linearly between sample points, and the intersection between that line and the displacement line is supposed to be the destination. For example, as shown in FIG. 12, a line is tentatively drawn between the two sample points Xa and Xb on the phase curve θ2(x), and the intersection X1′ between that line and the displacement line #1 is located. In the same way, a line is tentatively drawn between the two sample points Xd and Xe on the phase curve θ2(x), and the intersection X2′ between that line and the displacement line #2 is located. As shown in FIG. 12, the magnitude of displacement ΔX1 of the measuring point X1 is greater than that ΔX2 of the measuring point X2. That is to say, this object has expanded. As can be seen, according to the present invention, not just when the overall object travels horizontally as shown in FIG. 5 but also when the object compresses or expands in the wave propagating direction, the location of each measuring point can be tracked accurately.

It should be noted that the interpolation method for approximating a phase curve based on a group of phase data does not have be such a linear interpolation but may also be any other interpolation method that uses a curve or a spline curve by a minimum square method, for example.

Hereinafter, a location tracking method according to the present invention, including such digital signal processing, will be described with reference to FIGS. 12 and 13. First, in Step S351, an object is irradiated with a wave and the wave reflected from the object is converted into an electrical signal, thereby obtaining a received signal y1(x). Next, in Step S352, the received signal y1(x) is subjected to an orthogonal detection using a reference signal with a single frequency f to find the phase of the received signal y1(x) with respect to the reference signal. By making sampling either before or after the orthogonal detection, the resultant complex received signal turns into a group of digital complex data. The rest of the signal processing is performed digitally.

Subsequently, in Step S353, a group of phase data θ1(x) is calculated based on the group of complex data thus obtained. If no group of phase data has been obtained in the previous processing step, the arctangent of the group of complex data is calculated, thereby obtaining a group of phase data representing phases at respective sample points. On the other hand, if a group of phase data has been obtained in the previous processing step, another group of phase data is figured out by the method that has already been described with reference to FIG. 10 or 11.

Next, in Step S354, it is determined whether the location of the measuring point should be initialized or not. More particularly, if a signal instructing that the tracking location of the measuring point be initialized has been detected, then the measuring point is assigned to an initial location X1 in Step S358. Even if the received signal is the first one that has ever been received, the processing step S358 is also carried out in response to that signal instructing that the tracking location be initialized. Either a single measuring point or a plurality of measuring points may be set. Subsequently, in Step S359, the phase value of the initial location, which is the location of the measuring point on the group of phase data θ1(x), is calculated by the method described above. Thereafter, in Step S360, a displacement line (given by Equation (7)) that passes the initial location, which is the location of the measuring point on the group of phase data θ1(x), and that is defined by the frequency f of the reference signal and the propagation velocity C of the wave is determined.

The group of phase data θ1(x) thus obtained is stored as the previous group of phase data in a memory in Step S361. Also, the group of complex data obtained by the orthogonal detection is stored as the previous group of complex data in the memory in Step S362.

Next, the processing steps S351, S352 and S353 are carried out all over again. Specifically, the object is irradiated with another wave, a received signal is generated based on the wave reflected, and a group of phase data θ2(x) representing the phase of the received signal in the wave propagating direction with respect to the reference signal is obtained. The group of phase data θ2 is figured out based on the group of phase data θ1(x) by the method that has already been described with reference to FIG. 10 or 11. Unless the signal instructing that the tracking location of the measuring point be initialized has been received, the process advances from Step S354 to Step S355.

In Step S355, the intersection between the group of phase data θ2(x) and the displacement line is located just as described above. Then, in Step S356, the intersection thus found is defined as the destination of the measuring point. That is to say, the new location of the measuring point is determined. The group of phase data θ2(x) thus obtained is stored as the previous group of phase data in the memory in Step S361. Also, the group of complex data is stored as the previous group of complex data in the memory in Step S362.

After that, the same processing steps S351, S352, S353, S354, S355, S356, S361 and S362 are carried out all over again to irradiate the object with another wave, obtain another group of phase data, find an intersection between a displacement line and the group of phase data, and locate the measuring point repeatedly. Also, the signal instructing that the tracking location of the measuring point be initialized is output at an appropriate interval. Thus, every time the signal instructing that the tracking location of the measuring point be initialized is detected, the location of the measuring point is initialized and a displacement line that passes the initial location is obtained.

### Embodiment 2

According to the method of the first preferred embodiment described above, a displacement line is calculated only when the location of a measuring point should be initialized. That is to say, the displacement line is calculated based on a phase curve when the location of the measuring point is initialized. This means that to calculate the tracking location based on the intersection between the displacement line and the phase curve, the phase relation between the newest phase curve and the phase curve when the location of the measuring point was initialized needs to be maintained. That is why either by sequentially adding the phase difference at every sample point to the previous group of phase data as shown in FIG. 10 or adding the phase difference at one sample point to the previous sample point to calculate a reference sample point as shown in FIG. 11, the phase relation between the newest phase curve and the phase curve when the location of the measuring point was initialized is maintained. In other words, there are a number of phase curves between the newest phase curve and the phase curve when the location of the measuring point was initialized, and the accumulation of phase differences between those phase curves shows the phase relation between the newest phase curve and the phase curve when the location of the measuring point was initialized.

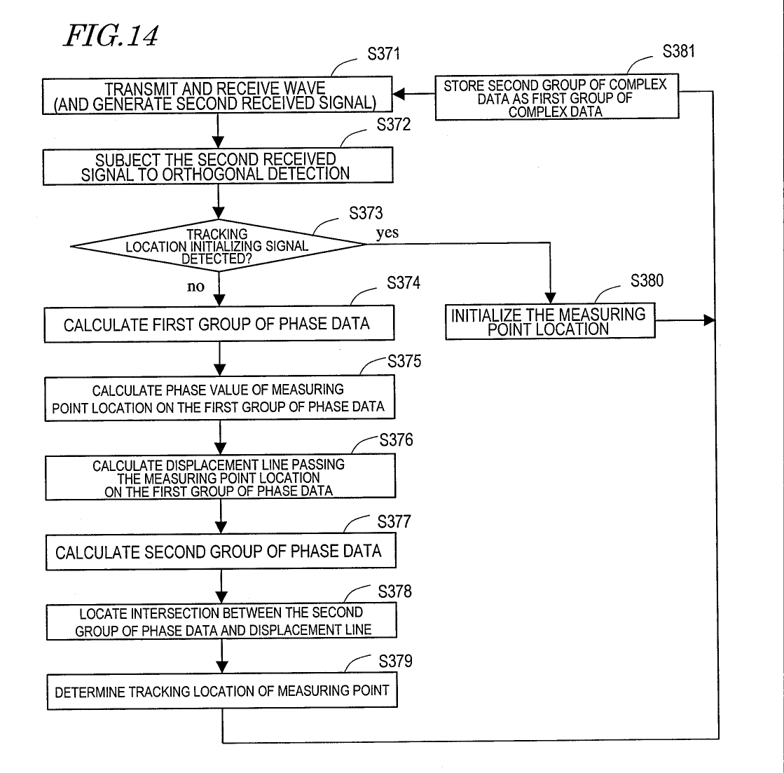

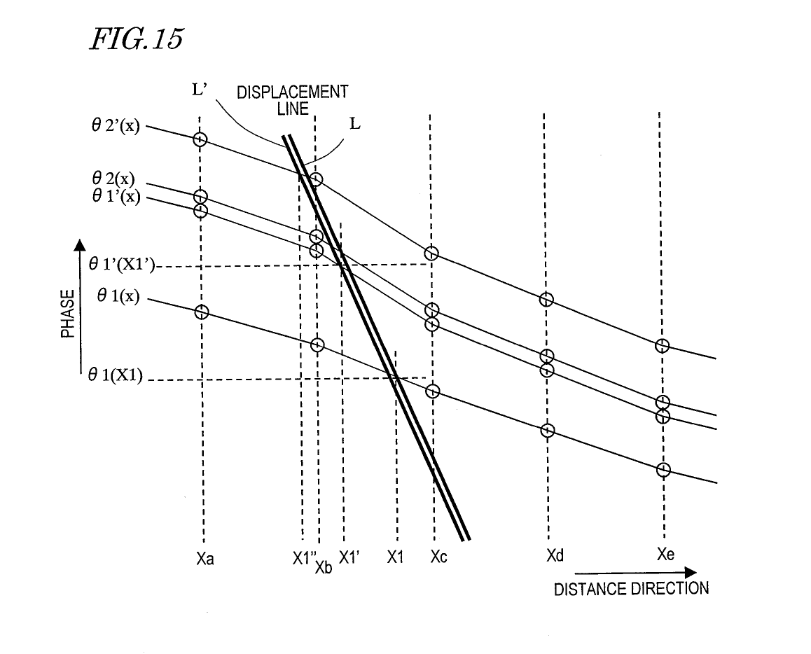

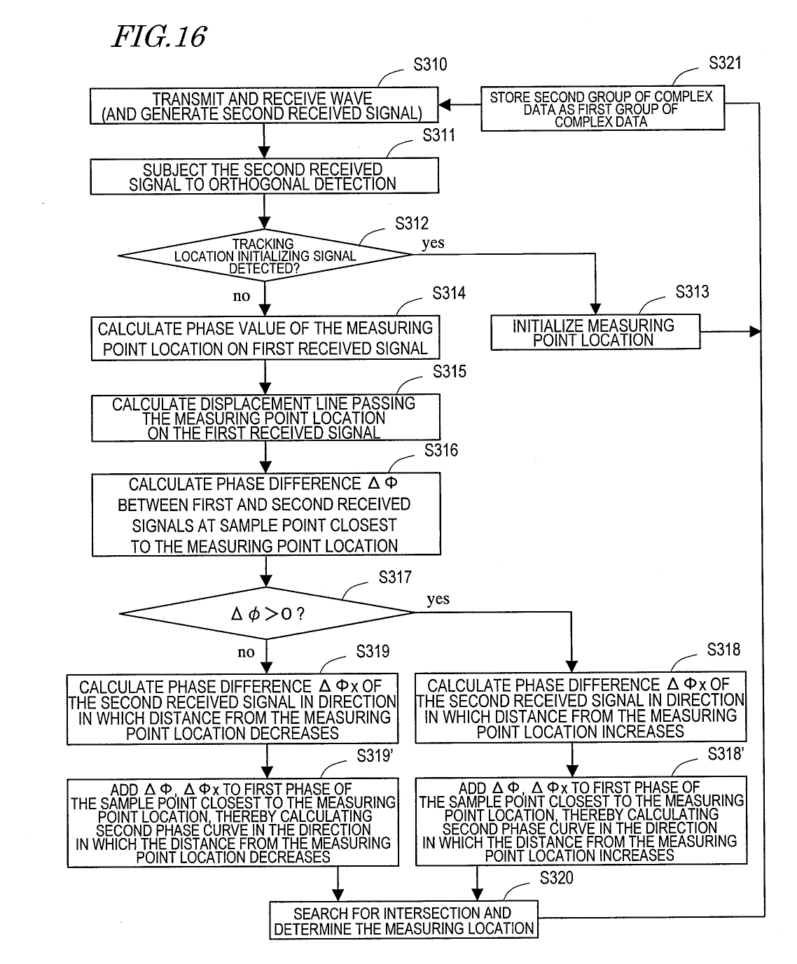

On the other hand, according to this preferred embodiment, every time a phase curve is calculated, a displacement line is calculated. Hereinafter, a location tracking method according to this preferred embodiment will be described with reference to FIGS. 14 and 15. In the following example, transmission of a wave and reception of a reflected wave are repeatedly performed, one of the resultant received signals that has just been obtained as a result of the newest transmission and reception will be referred to herein as a “second received signal” and another received signal that was obtained as a result of the previous transmission and reception a “first received signal”, respectively. The same naming rule will also apply to complex received signals, phase curves and so on obtained by detecting the received signals. If the signal processing is carried out digitally, the complex received signals and phase curves are groups of complex data and groups of phase data, respectively.

First, in Step S371, an object is irradiated with a wave and the wave reflected from the object is converted into an electrical signal, thereby obtaining a second received signal. Next, in Step S372, the second received signal is subjected to an orthogonal detection using a reference signal with a single frequency f to find the phase of the second received signal with respect to the reference signal. In this manner, a second complex received signal is obtained. Since digital signal processing is carried out in this preferred embodiment, sampling is performed either before or after the orthogonal detection, and the resultant second complex received signal is a second group of digital complex data.

Subsequently, in Step S373, it is determined whether the location of the measuring point should be initialized or not. More particularly, if a signal instructing that the tracking location of the measuring point be initialized has been detected, then the measuring point is assigned to an initial location in Step S380. Even if the received signal is the first one that has ever been received, the processing step S380 is also carried out in response to that signal instructing that the tracking location be initialized. In FIG. 15, the initial location of the measuring point is set at the distance X1. Next, Step S381 is carried out to store the second group of complex data as a first group of complex data in a memory.

Next, the processing steps S371 and S372 are carried out all over again. Specifically, the object is irradiated with another wave, a second received signal is generated based on the wave reflected, and the second received signal is subjected to the orthogonal detection to obtain a second group of complex data. Unless the signal instructing that the tracking location be initialized has been received, the process advances from Step S373 to Step S374.

In Step S374, the arctangent of the first group of complex data stored in the memory is calculated, thereby obtaining a first group of phase data (or a first phase curve). In FIG. 15, the first group of phase data is identified by θ1(x). Next, in Step S375, the phase θ1(X1) at the location X1 on the first group of phase data is calculated. Specifically, a line passing the sample points θ1(Xb) and θ1(Xc) before and after the location X1 is drawn tentatively and the phase θ1(X1) at X1 is calculated by linear interpolation.

Thereafter, in Step S376, a displacement line L that passes the measuring point (X1, θ1(X1)) on the first group of phase data and that has a gradient of −4πf/C is obtained on a distance-phase plane. Next, in Step S377, a second group of phase data θ2(x) is calculated based on the first group of phase data θ1(x) as already described for the first preferred embodiment with reference to FIG. 10 or 11.

Subsequently, in Step S378, the intersection between the second group of phase data θ2(x) and the displacement line is located just as already described with reference to FIG. 12. The intersection is identified by (X1′, θ2(X1′)) in FIG. 15. Next, in Step S379, the intersection thus found is defined as the destination of the measuring point. That is to say, the measuring point moves from X1 to X1′.

Thereafter, in Step S381, the second group of complex data is stored as the first group of complex data in the memory.

After that, the same processing steps S371 through S379 and S381 are carried out all over again to irradiate the object with another wave, generate a second received signal based on the wave reflected, subject the second received signal to an orthogonal detection and calculate the second group of complex data repeatedly. In Step S374, the arctangent of the first group of complex data stored in the memory is calculated, thereby obtaining a first group of phase data (or a first phase curve). In FIG. 15, the first group of phase data is identified by θ1′(x).

The first group of complex data that has been stored in the memory was formerly the second group of complex data that was used to calculate the previous second group of phase data θ2(x). However, the previous second group of phase data θ2(x) has been calculated based on the first group of phase data θ1(x) by the method shown in FIG. 10 or 11, whereas the first group of phase data θ1′(x) is obtained by calculating the arctangent of the first group of complex data stored in the memory. That is why these two groups may have different phases and the first group of phase data θ1′(x) does not always agree with the second group of phase data θ2(x).

Next, in Step S375, the phase θ1′(X1′) at the location X1′ on the first group of phase data θ1′(x) is calculated. Specifically, a line passing the sample points θ1′(Xb) and θ1′(Xc) before and after the location X1′ is drawn tentatively and the phase θ1′(X1′) at X1′ is calculated by linear interpolation.

Thereafter, in Step S376, a displacement line L′ that passes the measuring point (X1′, θ1′(X1′)) on the first group of phase data θ1′(x) and that has a gradient of −4πf/C is obtained on a distance-phase plane. Next, in Step S377, a second group of phase data θ2′(x) is calculated based on the first group of phase data θ1′(x) as described above.

Subsequently, in Step S378, the intersection between the second group of phase data θ2(x) and the displacement line is located just as already described with reference to FIG. 12. The intersection is identified by (X1″, θ2′(X1″)) in FIG. 15. Next, in Step S379, the intersection thus found is defined as the destination of the measuring point. That is to say, the measuring point moves from X1′ to X1″.

As described above, according to this preferred embodiment, each displacement line is re-calculated based on the previous phase curve. That is why there is no need to define the phase relation between the newest phase curve and the other phase curves that precede the previous one. As a result, it is possible to substantially avoid a situation where noise or error that has occurred halfway through a series of processing will have an unbeneficial effect for the rest of the processing.

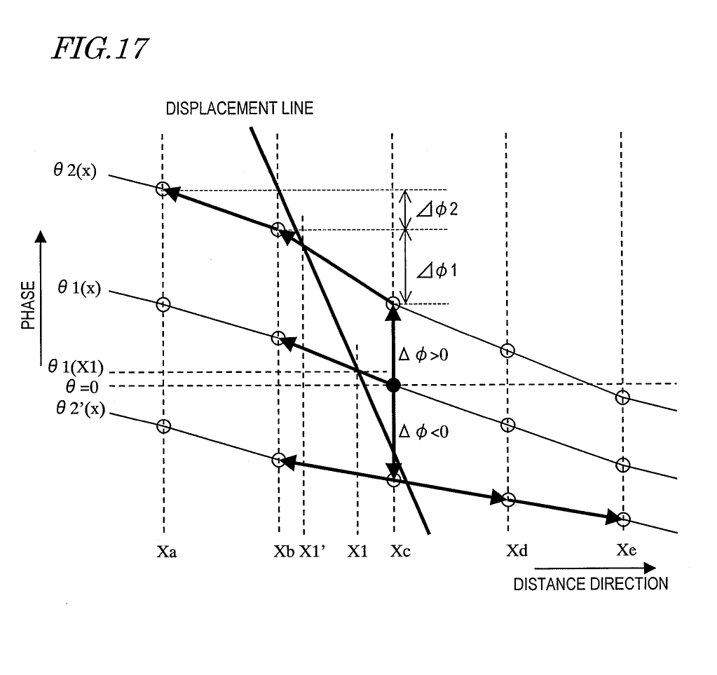

Hereinafter, a simplified version of the location tracking method according to this preferred embodiment will be described with reference to FIGS. 16 and 17. In the following example, transmission of a wave and reception of a reflected wave are also repeatedly performed, one of the resultant received signals that has just been obtained as a result of the newest transmission and reception will be referred to herein as a “second received signal” and another received signal that was obtained as a result of the previous transmission and reception a “first received signal”, respectively. The same naming rule will also apply to complex received signals, groups of complex data, phase curves, groups of phase data and so on obtained by detecting the received signals. First, in Step S310, an object is irradiated with a wave and the wave reflected from the object is converted into an electrical signal, thereby obtaining a second received signal. Next, in Step S311, the second received signal is subjected to an orthogonal detection using a reference signal with a single frequency f to find the phase of the second received signal with respect to the reference signal. In this manner, a second group of complex data is obtained.

Subsequently, in Step S312, it is determined whether the location of the measuring point should be initialized or not. More particularly, if a signal instructing that the tracking location of the measuring point be initialized has been detected, then the measuring point is assigned to an initial location in Step S313. Even if the received signal is the first one that has ever been received, the processing step S313 is also carried out in response to that signal instructing that the tracking location be initialized. In FIG. 17, the initial location of the measuring point is set at the distance X1. Next, Step S321 is carried out to store the second group of complex data as a first group of complex data in a memory.

Next, the processing steps S310, S311 and S312 are carried out all over again. Specifically, the object is irradiated with another wave, a second received signal is generated based on the wave reflected, and the second received signal is subjected to the orthogonal detection to obtain a second group of complex data. Unless the signal instructing that the tracking location be initialized has been received, the process advances from Step S312 to Step S314.

Next, in Step S314, the phase θ1(X1) at the location X1 on the first group of phase data is calculated based on the first group of complex data. Specifically, first, the arctangent of the group of complex data between the previous and following sample points Xb and Xc is calculated to obtain phase data θ1(Xb) and θ1(Xc). Next, a line is drawn tentatively between θ1(Xb) and θ1(Xc) and the phase θ1(X1) at X1 is calculated by linear interpolation. In this case, the phase θ1(Xc) at the sample point Xc that is closest to the location X1 of the measuring point may set equal to zero. Thereafter, in Step S315, a displacement line that passes (X1, θ1(X1)) and that has a gradient of −4πf/C is obtained on a distance-phase plane.

Subsequently, in Step S316, the phase difference Δbetween the phase data θ1(x) and the phase data θ2(x) or θ2′(x), which are the values of the first and second received signals at the sample point Xc that is closest to the measuring point location X1, is calculated. Specifically, the phase difference Δφ is figured out by calculating the arctangent of a complex conjugate product of the first and second groups of complex data at the sample point Xc. If the phase difference Δφ is positive as in Steps S317, S318 and S318′, the destination of the measuring point is located in the direction in which the distance decreases. In the example shown in FIG. 17, θ2(x) has such a pattern, and therefore, the intersection is searched for leftward from the measuring point X1. As shown in FIG. 17, the phase difference Δφ1 of the second group of phase data between the sample points Xc and Xb is calculated. Specifically, the phase difference Δφ1 is figured out by calculating the arctangent of a complex conjugate product of the second complex data at the sample points Xb and Xc. By adding the phase difference Δφ to the phase θ1(Xc) of the first group of phase data at the sample point Xc, the phase θ2(Xc) of the second group of phase data at the sample point Xc can be obtained. Also, by adding the phase difference Δφ1, the phase θ2(Xb) at the sample point Xb can be obtained.

A line is tentatively drawn between θ2(Xb) and θ2(Xc) and an intersection between that line and the displacement line is found. If there is no intersection between the sample points Xb and Xc, then the phase difference Δφ2 between the sample points Xb and Xa of the second received signal is figured out and added to the phase θ2(Xb), thereby calculating the phase θ2(Xa) at the sample point Xa. Specifically, a line is tentatively drawn between θ2(Xa) and θ2(Xb) and an intersection between that line and the displacement line is located. If there is no intersection between Xa and Xb, then the same type of processing is performed in the direction in which the distance further shortens, thus finding an intersection X1′. In Step S320, the location of the intersection X1′ thus found is defined as the location of the next measuring point.

If the phase difference Δφ is negative as in Steps S319 and S319′, the destination of the measuring point is located in the direction in which the distance increases. In the example shown in FIG. 17, θ2′(x) has such a pattern, and therefore, the second group of phase data θ2′(x) is calculated between Xb and Xc and then between Xc and Xd, and the intersection is searched for rightward from the measuring point X1. Then, in Step S320, the location of the intersection X1′ thus found is defined as the location of the next measuring point.

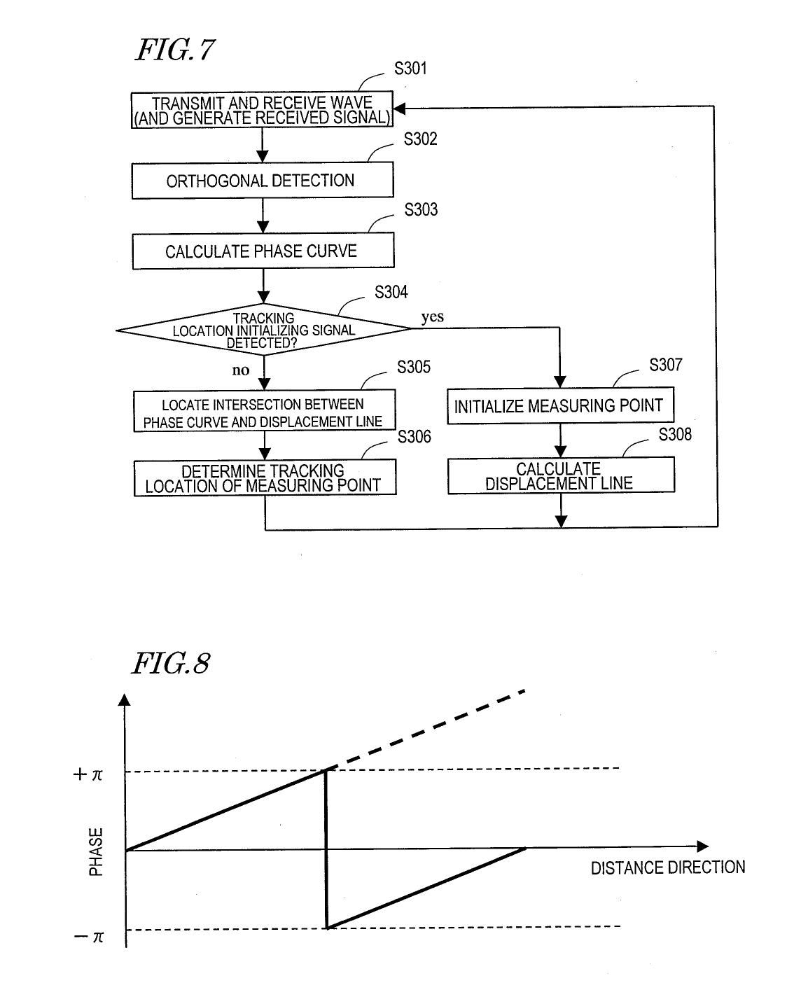

Next, the processing step S321 is carried out and the second group of complex data is stored as a first group of complex data in the memory. By adopting such a method, the destination of the measuring point can be located by calculating only required phases for just a portion of a phase curve according to the sign of the phase difference Δφ without calculating the phase at every sample point as shown in FIG. 7. As a result, the computational complexity can be reduced. Among other things, calculation of an arctangent, which requires a lot of computations, can be omitted.

According to the procedure that has just been described with reference to FIGS. 16 and 17, data at some of the sample points on a phase curve is figured out following the procedure shown in FIG. 11. If the first phase curve θ1(x) has been obtained to a certain extent, then the data at those sample points on the phase curve may be figured out following the procedure shown in FIG. 10 instead of that shown in FIG. 11.

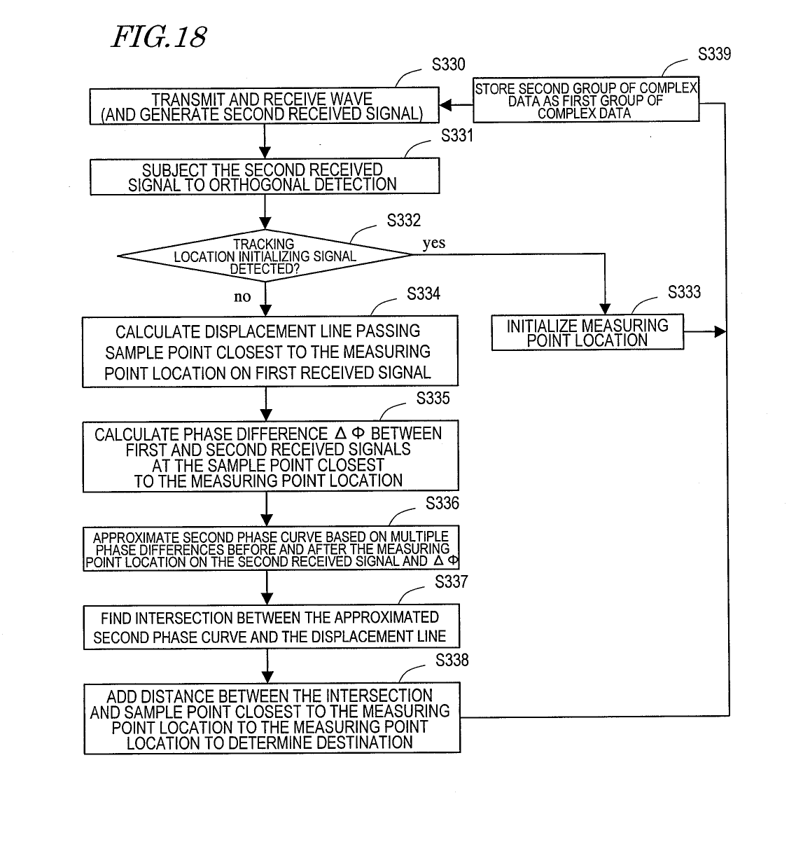

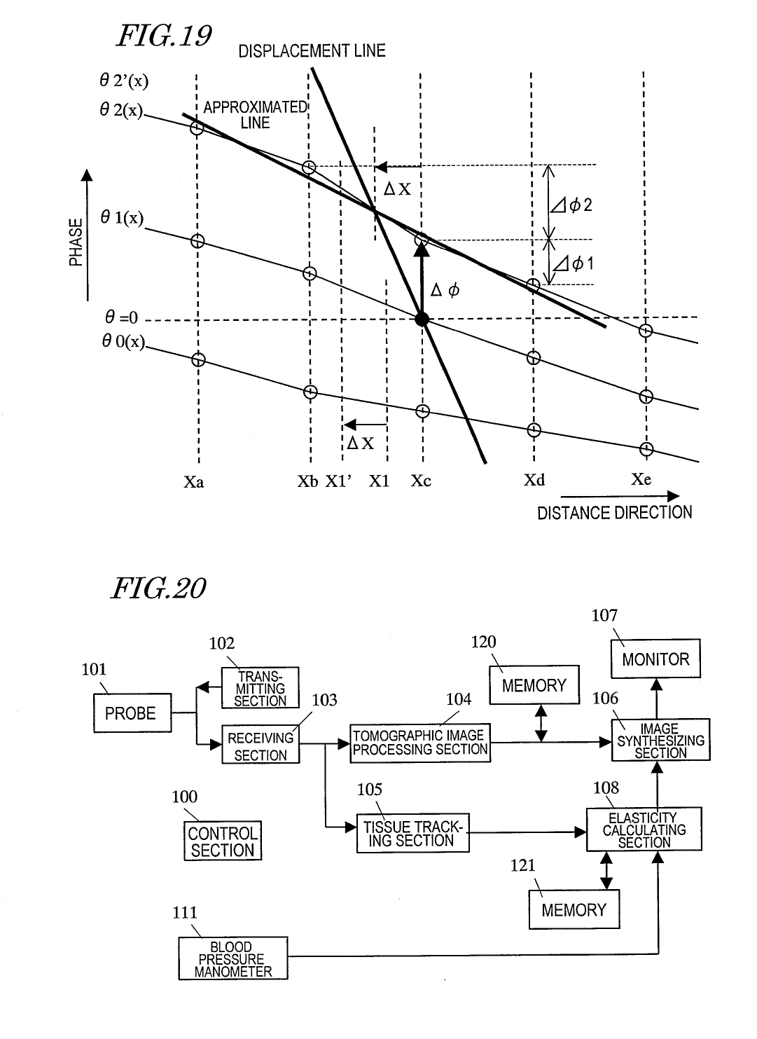

Hereinafter, a further simplified version of the location tracking method that has already been described with reference to FIGS. 16 and 17 will be described with reference to FIGS. 18 and 19. In the following example, transmission of a wave and reception of a reflected wave are also repeatedly performed, one of the resultant received signals that has just been obtained as a result of the newest transmission and reception will be referred to herein as a “second received signal” and another received signal that was obtained as a result of the previous transmission and reception a “first received signal”, respectively. The same naming rule will also apply to complex received signals, groups of complex data, phase curves, groups of phase data and so on obtained by detecting the received signals. First, in Step S330, an object is irradiated with a wave and the wave reflected from the object is converted into an electrical signal, thereby obtaining a second received signal. Next, in Step S331, the second received signal is subjected to an orthogonal detection using a reference signal with a single frequency f to find the phase of the second received signal with respect to the reference signal. In this manner, a second group of complex data is obtained.

Subsequently, in Step S332, it is determined whether the location of the measuring point should be initialized or not. More particularly, if a signal instructing that the tracking location of the measuring point be initialized has been detected, then the measuring point is assigned to an initial location in Step S333. Even if the received signal is the first one that has ever been received, the processing step S333 is also carried out in response to that signal instructing that the tracking location be initialized. In FIG. 19, the initial location of the measuring point is set at the distance X1. Next, Step S339 is carried out to store the second group of complex data as a first group of complex data in a memory.

Next, the processing steps S330, S331 and S332 are carried out all over again. Specifically, the object is irradiated with another wave, a second received signal is generated based on the wave reflected, and the second received signal is subjected to the orthogonal detection to obtain a second group of complex data. Unless the signal instructing that the tracking location be initialized has been received, the process advances from Step S332 to Step S334.

Thereafter, in Step S334, a displacement line, which passes the sample point (Xc, θ1(Xc)) that is closest to the measuring point location X1 on the first group of phase data and which has a gradient of −4πf/C, is obtained on a distance-phase plane. Next, a second group of phase data is calculated based on the second group of complex data and an intersection with the displacement line is calculated. Subsequently, in Step S335, the phase difference Δφ between the first and second groups of phase data at the sample point Xc that is closest to the location X1 is obtained. Then, in Step S336, the phase difference Δφ2 between the sample points Xc and Xb and the phase difference Δφ1 between the sample points Xc and Xd are calculated based on the second group of phase data. After that, an approximation line of the second group of phase data θ2(x) in the vicinity of Xc is figured out based on the phase differences Δφ, Δφ1 and Δφ2 by a minimum square method, for example.

Next, in Step S337, an intersection between that approximation line and the displacement line is located. Then, in Step S338, the distance ΔX between the intersection and the sample point Xc that is closest to the measuring point location X1 is calculated as the magnitude of displacement. By adding ΔX to the initial location X1, the next location X1′ of the measuring point can be obtained. Next, Step S339 is performed to store the second group of complex data as a first group of complex data in the memory. By repeatedly performing these processing steps S330, S331, S332, S334 through S338 and S339, the location of the measuring point can be tracked with the computational complexity further reduced. Among other things, calculation of an arctangent, which requires a lot of computations, can be omitted.

The approximation line does not have to be calculated by the minimum square method using three points but may also be calculated based on two points or more than three points. Besides, not just the linear approximation but also a high-order curve or any other curve may be adopted as well. Furthermore, phases may be selectively used according to the sign of Δφ. Specifically, if Δφ is positive, the destination is located in the direction in which the distance decreases, and therefore, θ2(Xa), θ2 (Xb) and θ2 (Xc) in the direction in which the distance from the previous location X1 decreases may be used. On the other hand, if Δφ is negative, the destination is located in the direction in which the distance increases, and therefore, θ2(Xb), θ2(Xc) and θ2(Xd) in the direction in which the distance from the previous location X1 increases may be used.

### Embodiment 3

The location tracking method of the present invention described above can be used as a method for tracking the location of any of various moving objects by using an acoustic wave, a radio wave, a light wave or a laser beam. Hereinafter, an ultrasonic diagnostic apparatus for evaluating a property of a subject's tissue (e.g., the elasticity thereof, among other things) by the location tracking method of the present invention will be described.

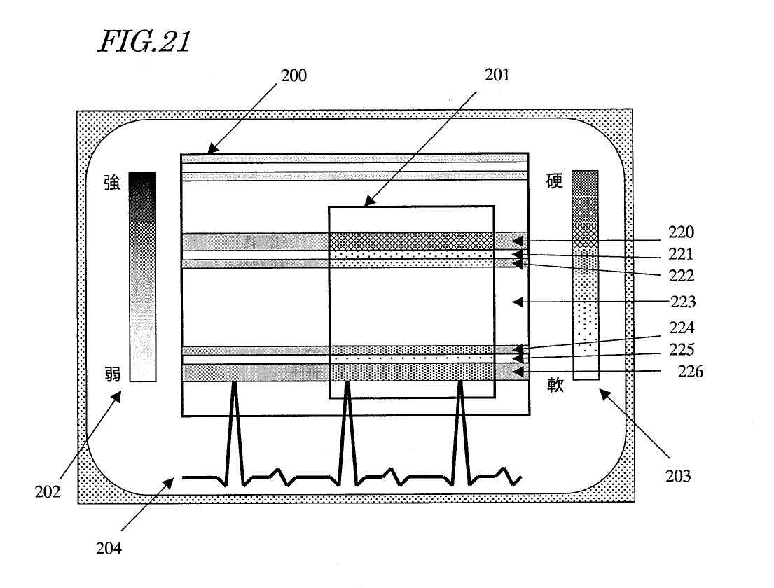

FIG. 20 is a block diagram showing a preferred embodiment of an ultrasonic diagnostic apparatus according to the present invention. As shown in FIG. 20, the ultrasonic diagnostic apparatus includes a transmitting section 102, a receiving section 103, a tomographic image processing section 104, a tissue tracking section 105, an image synthesizing section 106, an elasticity calculating section 108, and memories 120 and 121. The apparatus further includes a control section 100 for controlling all of these elements. Although not shown, an input device such as a keyboard, a track ball, a switch, a button or a key and an output device such as an LCD monitor are also connected to the control section 100.

In accordance with the instruction given by the control section 100, the transmitting section 102 generates a high-voltage signal that drives the probe 101 at a specified timing. The probe 101 converts the signal that has been generated by the transmitting section 102 into an ultrasonic wave and sends out the ultrasonic wave toward a subject, which is the object of measurement, and also detects an ultrasonic echo that has been reflected by an internal organ of the subject and converts the echo into an electrical signal. A number of piezoelectric transducers are arranged in the probe 101. By changing the piezoelectric transducers to use, the timing to apply a voltage to the piezoelectric transducers, or the voltages themselves, the probe 101 controls the scan line position, angle of deflection and focus of the ultrasonic waves to transmit and receive.

The receiving section 103 amplifies the received signal, and adds appropriate delays to the signals received from the respective piezoelectric transducers. In this manner, the receiving section 103 detects either only an ultrasonic wave that has been reflected from a predetermined point (i.e., a focused ultrasonic beam) or only an ultrasonic wave that has come from a predetermined direction (or at a predetermined angle of deflection). In the latter case, the receiving section 103 forms an ultrasonic beam so to speak.

The tomographic image processing section 104 includes a filter, a detector, a logarithmic amplifier and a scanning converter, and analyzes mainly the amplitude of the received signal, thereby presenting the internal structure of the subject as an image.

The tissue tracking section 105 operates in cooperation with the transmitting section 102 and the receiving section 103 to track the motions of multiple measuring points that have been set in the subject's tissue parallel to the ultrasonic wave transmitting and receiving direction, which is the acoustic line direction of the ultrasonic waves. The tissue tracking section 105 includes an ASIC, an FPGA, a DSP, a CPU, a memory and so on, stores a program for performing respective processing steps of the location tracking method of the present invention and reads and executes the program if necessary.

The blood pressure manometer 111 measures the blood pressure of the subject and outputs the blood pressure value thus obtained to the elasticity calculating section 108. The elasticity calculating section 108 calculates the magnitude of strain given by Equation (3) based on the motion of the subject's tissue tracked and the elasticity given by Equation (4) based on the magnitude of strain and blood pressure value, respectively, and outputs the elasticity as either a numerical value or a two-dimensional image. The image synthesizing section 106 synthesizes together at least the tomographic image and either the elasticity image or the elasticity value and then presents a synthetic image on the monitor 107. The memory 121 stores the tracking location information, i.e., one of the subject tissue's motion, magnitude of strain and elasticity, which is read in re-calculating the elasticity value when the transmission and reception of ultrasonic waves are stopped (which will be referred to herein as a “freeze state”). The memory 120 stores a tomographic image, which is read synchronously with the elasticity in the freeze state.

FIG. 21 illustrates a picture that may be presented on the monitor 107 and shows an exemplary result of measurement of the vascular wall's elasticity. In FIG. 21, a two-dimensional elasticity image 201, representing the distribution of elasticity at a site on the vascular wall in colors, is superimposed on a vascular wall's monochrome tomographic image 200 on the monitor. The two-dimensional elasticity image 201 sets the areas to monitor so as to include a vascular anterior wall adventitia 220, an anterior wall media 221, an anterior wall intima 222, a blood vessel lumen 223, a posterior wall intima 224, a posterior wall media 225, and a posterior wall adventitia 226.

In transmitting or receiving the ultrasonic wave (which will be referred to herein as a “live state”), the monochrome tomographic image 200 is updated at a rate of 10 frames per second as in a conventional ultrasonic diagnostic apparatus. Meanwhile, the elasticity image 201 is updated once every cardiac cycle. In the freeze state, the subject's motion information or magnitude of strain or elasticity is read from the memory 121, the elasticity is re-calculated based on that data and displayed, and a tomographic image is read from the memory 120 and presented synchronously with the elasticity at that time.

The monochrome tomographic image 200 is displayed at monochromatic gray scales corresponding to the reflection intensities along with a scale 202 indicating the reflection intensities. On the other hand, the elasticity image 201 is displayed in color tones corresponding to the elasticity values along with a scale 203 indicating the elasticity values. Also displayed under the monochrome tomographic image 200 is a biomedical signal waveform 204 such as an electrocardiogram.

According to the present invention, the location of a measuring point that has been set in a subject can be tracked precisely, and therefore, the elasticities of respective portions of the subject and their distribution can be obtained accurately. Thus, by using the ultrasonic diagnostic apparatus of the present invention, the degree of advancement of arterial sclerosis on a vascular wall and its distribution can be measured or a disease that has been produced on the arterial vascular wall can be spotted.

Since the subject's tissue can be tracked accurately by the tracking method of the present invention, the values to be measured eventually are not limited to elasticity and magnitude of strain but may also include other measuring indices such as viscosity μ, intima-media thickness (IMT) for use in inspecting arterial sclerosis and a local pulse wave velocity (PWV) and the cardiac contraction and dilation function. All of these indices can also be measured accurately.

Furthermore, the location tracking method of the present invention can be used not just in a medical ultrasonic diagnostic apparatus but also in various other fields as a method for tracking the location of any of numerous other objects of measurement.

The location tracking method of the present invention is applicable to any type of object tracking device that uses any of various waves such as a flaw detector, a nondestructive tester, a fish finder and a sonar that use ultrasonic waves, an aircraft control radar, a weather observation radar, and a military radar that use electromagnetic waves, and a chartometer and a displacement meter that use visible radiation, infrared rays and ultraviolet laser beams.

### INDUSTRIAL APPLICABILITY

The location tracking method of the present invention can be used not just in the ultrasonic diagnostic apparatus described above but also in various other fields to track the location of any of numerous types of objects of measurement.

1. A location tracking method for tracking the motion of a measuring point that has been set on an object of measurement by repeatedly irradiating the object with waves and by analyzing received signals that are based on the waves reflected from the object, the method comprising the steps of:
(A) irradiating the object with a wave and calculating an initial phase curve showing a phase shift of a received signal in a propagation direction of the wave with respect to a reference signal, the received signal being based on the wave reflected;
(B) setting an initial location in the wave propagating direction and assigning the measuring point to the initial location;
(C) defining a displacement line, which passes the initial location on the initial phase curve and of which the gradient is calculated based on the frequency of the reference signal; and
(D) irradiating the object with the waves a number of times, thereby calculating phase curves, each representing a phase shift of the received signal with respect to the reference signal, and defining the intersection between the displacement line and each said phase curve as a location to which the measuring point has been displaced.

2. The location tracking method of claim 1, wherein the received signal is discrete quantized digital data, and wherein the step (D) includes calculating the newest phase curve by figuring out a phase difference between the newest received signal and the signal that has been received the previous time and by adding the phase difference thus obtained to the previous phase curve.

3. The location tracking method of claim 1, wherein the received signal is discrete quantized digital data, and wherein the step (D) includes calculating the newest phase curve by figuring out a phase difference between the newest received signal and the signal that has been received the previous time at one of multiple sample points that have been set in the wave propagating direction, adding the phase difference thus obtained to a phase at the one sample point on the previous phase curve, and sequentially adding the phase difference of the newest received signal in a depth direction to the sum.

4. The location tracking method of claim 1, wherein the received signal is discrete quantized digital data, and wherein the step (C) includes determining a phase at the initial location by subjecting sampled data values of multiple phases to interpolation.

5. The location tracking method of claim 1, wherein the received signal is discrete quantized digital data, and wherein the step (D) includes finding an intersection either between multiple approximation lines, obtained based on sampled data of multiple phases, or between an approximation curve and the displacement line.

6. A location tracking method for tracking the motion of a measuring point that has been set on an object of measurement by repeatedly irradiating the object with waves and by analyzing received signals that are based on the waves reflected from the object, the method comprising the steps of:
(A) irradiating the object with a wave to obtain a first received signal that is based on the wave reflected;
(B) calculating a first phase curve showing a phase shift of the first received signal in a propagation direction of the wave with respect to a reference signal;
(C) setting a first location in the wave propagating direction and assigning the measuring point to the first location;
(D) defining a displacement line, which passes the first location on the first phase curve and of which the gradient is calculated based on the frequency of the reference signal;
(E) irradiating the object with a wave, thereby calculating a second phase curve, representing a phase shift of the second received signal with respect to the reference signal; and
(F) defining the intersection between the displacement line and the second phase curve as a second location to which the measuring point has been displaced,
wherein the location of the measuring point is tracked by performing the steps (B) through (E) all over again with the second received signal of the step (E) and the second location of the step (F) substituted for the first received signal and the first location, respectively.

7. The location tracking method of claim 6, wherein the step (E) includes calculating the second phase curve by figuring out a phase difference between the first and second received signals and by adding the phase difference thus obtained to the first phase curve.

8. The location tracking method of claim 6, wherein the step (E) includes calculating the second phase curve by figuring out a phase difference between the first and second received signals at one of multiple sample points that have been set in the wave propagating direction, adding the phase difference thus obtained to a phase at the one sample point on the first phase curve, and sequentially adding the phase difference of the second received signal in a depth direction to the sum.

9. The location tracking method of claim 8, wherein the step (E) includes setting one of the sample points closest to the measuring point.

10. The location tracking method of claim 9, wherein the step (E) includes calculating a portion of the second phase curve according to the sign of the phase difference between the first and second received signals and defining the intersection between the displacement line and that portion of the second phase curve as the location to which the measuring point has been displaced.

11. The location tracking method of claim 6, wherein the step (B) includes calculating the first phase curve based on only the first received signal.

12. The location tracking method of claim 6, wherein the step (B) includes calculating a portion of the first phase curve by finding phases at multiple sample points that have been set in the wave propagating direction.

13. A location tracking method for tracking the motion of a measuring point that has been set on an object of measurement by repeatedly irradiating the object with waves and by analyzing received signals that are based on the waves reflected from the object, the method comprising the steps of:
(A) irradiating the object with a wave to obtain a first received signal that is based on the wave reflected;
(B) calculating a first phase curve showing a phase shift of the first received signal in a propagation direction of the wave with respect to a reference signal;
(C) setting a first location in the wave propagating direction and assigning the measuring point to the first location;
(D) defining a displacement line, which passes one of multiple sample points that have been set in the wave propagating direction and of which the gradient is calculated based on the frequency of the reference signal, the one sample point being located closest to the first location;
(E) irradiating the object with a wave, thereby calculating a second phase curve representing a phase shift of the second received signal with respect to the reference signal; and
(F) finding the intersection between the displacement line and the second phase curve, adding the distance from the intersection to the sample point that is closest to the first location to the location of the measuring point to set a second location, and defining the second location as a location to which the measuring point has been displaced,
wherein the location of the measuring point is tracked by performing the steps (B) through (F) all over again with the second received signal of the step (E) and the second location of the step (F) substituted for the first received signal and the first location, respectively.

14. The location tracking method of claim 13, wherein the step (E) includes the steps of:
(E1) calculating the phase difference between the first and second received signals at the sample point that is located closest to the first location;
(E2) adding the phase difference, calculated in the step (E1), to the phase at the closest sample point on the first phase curve, thereby calculating the phase of the second received signal at that closest sample point;
(E3) calculating phase differences for multiple sample points that are adjacent to the closest sample point; and
(E4) defining either an approximation line or an approximation curve as the second phase curve based on the phase that has been calculated in the step (E2) and the phase difference that has been calculated in the step (E3).

15. The location tracking method of claim 1, wherein if the unit of the distance axis of a distance-phase plane is a length unit, the gradient of the displacement line is calculated −4πf/C, where f is the frequency of the reference signal and C is the propagation velocity of the waves.

16. The location tracking method of claim 1, wherein if the unit of the distance axis of a distance-phase plane is a receiving time unit, the gradient of the displacement line is calculated −2πf, where f is the frequency of the reference signal.

17. An apparatus for tracking the location of an object of measurement, the apparatus comprising:
a transmitting section for irradiating the object with a wave;
a receiving section for receiving a wave that has been reflected from the object to generate a received signal; and
a tracking section for tracking the location of a measuring point that has been set on the object by the method of claim 1 in cooperation with the transmitting and receiving sections.

18. An ultrasonic diagnostic apparatus comprising:
a transmitting section for transmitting an ultrasonic wave toward an object of measurement using a probe;
a receiving section for receiving a wave that has been reflected from the object through the probe to generate a received signal; and
a tracking section for tracking the location of a measuring point that has been set on the object by the method of claim 1 in cooperation with the transmitting and receiving sections,
wherein the apparatus evaluates at least one of the shape property and the attribute property of the object based on the location of the measuring point that has been calculated by the tracking section.

19. The ultrasonic diagnostic apparatus of claim 18, wherein the attribute property is one of the magnitude of strain, elasticity and viscosity of the object.

20. The ultrasonic diagnostic apparatus of claim 18, further comprising an elasticity calculating section that receives a signal representing a variation in stress applied to the object and that calculates the elasticity of the object based on the location of the measuring point that has been detected by the tracking section.

21. The ultrasonic diagnostic apparatus of claim 20, wherein the object of measurement is a vascular wall and the signal representing the variation in stress is a blood pressure waveform.

22. The ultrasonic diagnostic apparatus of claim 18, wherein the object of measurement is a vascular wall and the attribute property is a diagnostic index of arterial sclerosis including at least one of an intima-media thickness (IMT) and a pulse wave velocity (PWV).

23. The ultrasonic diagnostic apparatus of claim 18, wherein the object of measurement is a heart and the shape property is a contraction/dilation property.
